(19) United States
(12) Patent Application Publication
(10) Pub. No.: US 2021/0138090 A1
(43) Pub. Date: May 13, 2021

(54) uPAR targeting peptide for use in peroperative optical imaging of invasive cancer

(71) Applicant: FLUOGUIDE A/S, Copenhagen N (DK)

(72) Inventors: Andreas KJAER, Frederiksberg (DK); Morten PERSSON, Copenhagen (DK); Karina JUHL, Copenhagen N (DK); Sorel KURBEGOVIC, Copenhagen N (DK); Michael PLOUG, Copenhagen N (DK); Knud Jørgen JENSEN, Copenhagen Ø (DK); Kasper Kildegaard SØRENSEN, Smørumnedre (DK); Anders CHRISTENSEN, Lyngby (DK); Line HARTVIG, Hørsholm (DK); Grethe Nørskov RASMUSSEN, Farum (DK); Morten ALBRECHTSEN, Charlottenlund (DK)

(73) Assignee: FLUOGUIDE A/S, Copenhagen N (DK)

(21) Appl. No.: 17/150,133

(22) Filed: Jan. 15, 2021

Related U.S. Application Data
(63) Continuation-in-part of application No. 16/142,977, filed on Sep. 26, 2018.
Application No. 16/142,977 is a continuation of application No. 15/512,276, filed on Mar. 17, 2017, now Pat. No. 10,111,969.

(30) Foreign Application Priority Data
Sep. 17, 2014 (DK) PA 2014 70573

(51) Int. Cl. A61K 49/00

(52) U.S. Cl. CPC A61K 49/0056; A61K 49/0034; A61K 49/0032

(57) ABSTRACT

There is provided a novel conjugate that binds to the cell surface receptor uPA (uPAR). The conjugate is based on a fluorescence-labeled peptide useful as a diagnostic probe to the surfaces of cells expressing uPAR. The conjugate is capable of carrying a suitable detectable and imageable label that will allow qualitative detection and also quantitation of uPAR levels in vitro and in vivo. This renders the surgical resection of tumors more optimal.

### CROSS REFERENCE TO RELATED APPLICATIONS

This application is a Continuation-in-Part of U.S. application Ser. No. 16/142,977, filed 26 Sep. 2018, which is a Continuation of U.S. application Ser. No. 15/512,276, filed 17 Mar. 2017, now issued U.S. Pat. No. 10,111,969, which is a National Stage of PCT/DK2015/050261, filed 3 Sep. 2015, which claims benefit of Serial No. PA 2014 70573, filed 17 Sep. 2014 and which applications are incorporated herein by reference. To the extent appropriate, a claim of priority is made to each of the above disclosed applications.

### FIELD OF THE INVENTION

The present invention relates to a novel conjugate that binds to the cell surface receptor urokinase-type plasminogen activator receptor (uPAR). More specifically the conjugate is based on a fluorescence-labeled peptide useful as a diagnostic probe to the surfaces of cells expressing uPAR. The conjugate of the invention is capable of carrying a suitable detectable and imageable label that will allow for clear tumor delineation both in vitro and in vivo. This renders the surgical resection of tumors more optimal.

### BACKGROUND OF THE INVENTION

When performing cancer surgery with intent of radically remove cancer and metastases, delineation of active tumour is a major challenge and accordingly, either cancer tissue is left behind with poor prognosis or to ensure radical surgery, unnecessary extensive surgery is performed with removal of substantial amounts of healthy tissue.

Developments in the area of improved methods for cancer resection have in many years been stagnant. A surgeon's finest task is still to differentiate between healthy and diseased tissue under white light illumination. This can in many cases be difficult due to hidden areas of diseased tissue. In cancer treatment the best prognosis comes with complete removal of the cancerous tissue [1, 2]. Today the gold standard for assuring optimal resection is to take histological samples in the tumor bed and test for positive tumour margins. Several studies have shown this to be both inaccurate and time consuming.

Intraoperative optical imaging is a new emerging technique that allows the surgeon to differentiate between healthy and diseased tissue with help from a targeted optical probe [3, 4]. Near Infrared (NIR) florescence-imaging is a newer technique that can be used in intraoperative optical imaging. NIR fluorescence has some advantages compared to other widely used fluorophores with lower wavelength maxima. Tissue penetration is one of the forces of NIR fluorophores (NIRFs). Moreover, tissue autoflourescence is minimised in the NIR range and therefore enhance the tumour to background ratio needed for intraoperative imaging. These properties make NIRFs ideal for intraoperative surgery.

In neurosurgical oncology, fluorescence to guide surgery of high-grade glioblastoma has already been investigated [1]. The current fluorescence guided surgery (FGS) use ALA induces PpIX fluorescence which utilise the PpIX produced in all mammal cells. However, a significant higher production of PpIX is found in tumour cells (14-17 pogue et all 2010). Even though this system delineates the tumour with success, the system still has its drawbacks. Therefore, a clear clinical need for more specific targeting with NIRFs has evolved.

Urokinase-type plasminogen activator receptor (uPAR) is frequently over expressed in many cancer types. Expression of uPAR is associated with metastatic disease and poor prognosis and the receptor is often located in excess in the invasive front of the tumour. This makes uPAR ideal as a targeted probe for intraoperative optical imaging. A well validated uPAR targeted peptide AE105 has been used extensively in PET imaging for targeting uPAR previously by our group [5-8].

Recently, optical imaging using fluorescence was introduced to help delineating tumors. One example is indocyanin green (ICG) that to some extent unspecifically leaks out into tumors due to vascularization and leaky vessels. However, the unspecific nature of the methods limits its value.

Handgraaf et al [15] recognize that ICG is a non-targeted dye and its chemical structure does not allow conjugation to tumor specific ligands.

WO2014/086364 and WO2013/167130 disclose the use of radionuclide-labelled uPAR binding peptides for PET-imaging of cancer diseases. Such compounds were coupled via a chelating agent to a radionuclide.

Hence, there is a need for an improved imaging probe for guided surgery.

### SUMMARY OF THE INVENTION

The present inventors have surprisingly conjugated AE105 with indocyanine green (ICG). Due to the relatively large size and high hydrophobicity of ICG, two glutamic acid was used as a linker between AE105 (Asp-([beta]-cyclohexyl-L-alanine (Cha))-Phe-(D)Ser-(D)Arg-Tyr-Leu-Trp-Ser) and ICG (FIG. 1), thus providing minimal interference between AE105 and ICG. This novel fluorescent probe AE105-Glu-Glu-ICG has unexpectedly shown both in vitro and in vivo potential for use in fluorescent-guided cancer resection. It is to be noted that the prior art does not focus on the fluorophore labelled uPAR-targeting peptide conjugate although the prior art discloses radionuclide-labelled uPAR binding peptides.

Accordingly, the novel probe AE105-Glu-Glu-ICG enables a whole new concept where targeted optical imaging of the invasive cancer cells uses the proteolytic system receptor uPAR as a target. The major advantages are that it is tumour specific and that it particularly accumulates in the invasive front of cancers. Accordingly, it is clearly indicating where the active border of a tumour is relative to surrounding healthy tissue. In this way, the surgeon can exactly see where the tumour stops and remove only the tumour. If no tissue lightening up is left behind the cancer was successfully removed.

In accordance with the present invention there is therefore inter alia provided a novel fluorophore labelled uPAR-targeting peptide conjugate having the formula:

X-Y-(Asp)-([beta]-cyclohexyl-L-alanine (Cha))-(Phe)-(D-Ser)-(D-Arg)-(Tyr)-(Leu)-(Trp)-(Ser) wherein,

X represents imageable moiety capable of detection either directly or indirectly in an optical imaging procedure, and
Y represents a spacer, a biomodifier or is absent.

Particularly preferred are conjugates having the formula

[chemical structure]

Other preferred alternatives are provided below.

The compounds are preferably for use in fluorescence guided surgical resection of tumours. In this respect the compounds are administered to a subject in a dose of 0.1-2,000 mg per person. In such an application it is very suitable for peroperative optical imaging of cancer.

The present invention also provides a pharmaceutical composition for optical imaging of cancer, wherein the composition comprises a compound of the invention together with at least one pharmaceutically acceptable carrier or excipient. The dose of the compound is preferably 0.1-2,000 mg per person.

The invention also encompasses the use of the compound for the manufacture of a diagnostic agent for use in a method of optical imaging of metastatic cancer involving administration of said compound to a subject and generation of an image of at least part of said subject.

In a further aspect there is provided a method of optical imaging of cancer of a subject involving administering the compound of the present invention to the subject and generating an optical image of at least a part of the subject to which said compound has distributed.

### BRIEF DESCRIPTION OF THE DRAWINGS

FIG. 1 shows the structural formula of the compound of the present invention with indications of peptide and fluorophore part.

FIG. 2 shows staining experiments with rabbit-anti-uPAR.

FIG. 3 shows photographs of tumor scans with the compound of the invention and with ICG.

FIG. 4 shows quantitative analysis of the tumor and background uptake.

FIG. 5 shows photographs of tumor scans with the compound of the invention using Fluorobeam®.

FIG. 6 illustrates the molecular structure of the uPAR-targeting peptide conjugate, IRDye800CW-AE344.

FIG. 7 illustrates the Surface Plasmon Resonance analysis of the affinity to uPAR of IRDye800CW-AE344, the top line (1) represents IRDye800CW-AE344: uPAR^{wt} and the bottom line (2) represents AE105: uPAR^{wt}.

FIG. 8 illustrates the spectral analysis of the probe IRDye800CW-AE344 demonstration absorption peak at 777 nm (full line), excitation peak at 784 nm (dotted line), and emission peak at 794 nm (dashed line) resulting in a Stokes shift of 10 nm.

FIG. 9 illustrates the photostability of IRDye800CW-AE344 after continuous laser exposure.

FIG. 10 illustrates the in vivo imaging specificity of IRDye800CW-AE344 in an orthotopic GBM. The upper row shows intact brain (NIR image exposure time 500 ms), whereas the lower row shows cross sectioned brain (NIR image exposure time 333 ms), (animal id 029, 6 nmol, 3 h).

FIG. 11 illustrates histology of brain with H&E staining, NIR microscopy, H&E and NIR merged, and immunohistochemical staining of uPAR. The arrow is pointing at a small leptomeningeal metastasis at the basis of the brain and is visible both on H&E staining and NIR microscopy (12 nmol IRDye800CW-AE344, 24 h).

FIG. 12 illustrates ex vivo dynamic NIR imaging of GBM on cross sectioned brain. The arrows in the pictures indicate the highest TBR (max) for the given dose of IRDye800CW-AE344 (all data are based on images with an exposure time of 333 ms).

FIG. 13 illustrates tumor-to-background ratio for different doses of IRDye800CW-AE344, the lines represents the doses 1 mmol (●), 3 nmol (▪), 6 nmol (▴), and 12 nmol (v), respectively.

FIG. 14 illustrates tumor mean fluorescence intensities (MFI) for different doses of IRDye800CW-AE344, the different bars represents the doses 1 nmol, 3 nmol, 6 nmol, and 12 nmol, respectively.

FIG. 15 illustrates tumor and background mean fluorescence intensities at 6 nmols IRDye800CW-AE344 in comparison with the TBR, the light gray (left) bar represents tumor, the dark gray (right) bar represents background and the line represents TBR.

FIG. 16 illustrates NIR images of cross sectioned brains at 1 h after injection of (left to right): 3 nmols IRDye800CW-AE344 (active), 3 nM IRDye800CW-AE344+600 nmols AE120 (blocked), and 3 nM IRDye800CW-AE354 (mutated).

FIG. 17 illustrates normalized TBRs (ref: active probe). The black bar represents IRDye800CW-AE344 (active), the light gray bar represents AE120 (blocked) and the dark gray bar represents IRDye800CW-AE354 (mutated).

FIG. 18 illustrates images of organs representing the fluorescence intensity and biodistribution of 3 nmols IRDye800CW-AE344 at 1 h post injection (exposure time: 333 ms). The arrow at the small intestines indicates the proximal end. Kidneys oversaturated and thus out of range at the color calibration bar.

FIG. 19 illustrates quantification of fluorescence signal for IRDye800CW-AE344. Left y axis represents the mean fluorescence intensity. Right y axis represents the relative biodistribution. Further, the black bars represent the mean signal intensity (a.u.) and the grey bars represent the relative biodistribution (the data are normalized with the skin as the reference due to highest uptake).

FIG. 20 illustrates images (white light, NIR and merged).

FIG. 21A shows NIR images of cross sectioned brains at 1 h after injection with mean fluorescence intensities in FIG. 21B for tumor and background and the corresponding normalized TBR values in FIG. 21C.

FIG. 22 there is shown biodistribution and acute toxicity with all data at 1 h post injection.

### DETAILED DESCRIPTION OF THE INVENTION

Concerning the synthesis of some of the peptides used in the present invention reference is made to U.S. Pat. No. 7,026,282.

### One First Example

The peptide AE105 (Asp-Cha-Phe-(D)Ser-(D)Arg-Tyr-Leu-Trp-Ser-OH) was synthesized by standard solid-phase peptide chemistry. The peptide AE105 was conjugated to ICG (4-(2-((1E,3E,5E,7Z)-7-(3(5-carboxypentyl)-1,1-dimethyl-1H-benzo[e]indol-2(3H)-ydlidene)hepta-1,3,5-trienyl)-1,1dimethyl-1H-benzo[e]indolium-3-yl)butane-1-sulfonate) with two glutamic acids as linker (ICG-Glu-Glu-AE105), see FIG. 1. The probe has a final weight of 2197.55 g/mol. For in vivo injection ICG-Glu-Glu-AE105 was dissolved in (2-hydroxypropyl)-β-cyclodextrin with 2% DSMO.

Cell Lines

Human glioblastoma cell line U87MG was purchased from the American Type Culture Collection and culture media was obtained from Invitrogen. U87MG was cultured in DMEM added 10% FBS and 1% PenStrep. When the cells reached 70-80% confluency the cells were harvested.

All animal experiments were performed under a protocol approved by the Animal Research Committee of the Danish Ministry of Justice. 5*10⁶ U87MG cells were suspended in 200 μl PBS and inoculated on both flanks of the mouse. When the tumours reached an appropriate size the mice were imaged with AE105-Glu-Glu-ICG.

Flowcytometry

After harvesting of cells were washed in buffer and stained with either an in-house produced antibody (3 μg/ml), IgG isotype (3 g/ml; 14-4714 eBioscience) or blank control for 1 hr in 4° C. on a shaking table. The cells were washed 3 times with buffer and then stained with a secondary antibody (goat-anti-mouse-PE 1/500;) for 30 min in 4° C. on a shaking table. The result was analysed on the BD FACSCanto cell analyser.

ELISA Assay

Tumours were homogenised and a suspension containing the tumor lysate were stored at −80° C. The plate was coated with an anti uPAR antibody R2 (3 μg/ml) overnight at 4° C. After this incubation 2% BSA was added for 5 min and the plate was washed with buffer. uPAR standard (10 ng/ml) or tumor lysate (diluted 1:20) was added and incubated for 2 hr in RT and washed with buffer. A primary antibody (rabbit-anti-uPAR, 1 μg/ml) was added to the well and incubated for 30 min in RT and washed. A secondary HRP conjugated anti-rabbit antibody was added (diluted 1:2500) and incubated for 30 min in RT and washed. The bound HRP conjugated antibody was quantified by adding 4 OPD tablets (Dako #S2045) in 12 ml water and 10 μl H₂O₂. The reaction was stopped with 1M H₂SO₄ when the proper coloration of the well was present. An ELISA reader was used to analyze the plate at 490 nm and 650 nm as reference.

Optical Imaging

The mice were injected with 10 nmol of AE105-Glu-Glu-ICG or ICG i.v., and imaged 15 hr post injection. Before scan the mice were anaesthetized with 2% isofluran and positioned in a prone position. For imaging the IVIS Lumina XR and the acquisition software Living Image were used. The excitation filter was set to 710 nm and the emission filter was set in the ICG position. Acquisition was set to auto-settings to achieve the best acquisition as possible.

After imaging with IVIS Lumina XR the mouse was moved to a Fluobeam setup and imaged with appropriate acquisition time.

The TBR values were calculated by drawing a ROI over each tumor and place the background ROI in an area with constant background signal.

Results

In the production of the novel uPAR targeted fluorescence probe of the present invention two glutamic acids were introduced as linkers to partly reduce a potential interaction between ICG and the binding affinity of AE105 toward uPAR. The results indeed revealed a reduction in the binding affinity towards purified uPAR for ICG-Glu-Glu-AE105 (IC₅₀≈80 nM) compared to AE105 (IC₅₀≈10 nM), however the probe surprisingly retained sufficient affinity for guided surgical procedures.

Before any in vivo experiments were initiated, with U87MG cancer cells the expression of uPAR was measured in vitro by flowcytometry. The staining with rabbit-anti-uPAR showed a clear rightshift in fluorescence compared to the control, thus confirming high level of uPAR expression (FIG. 2a). The expression of uPAR was also investigated on histological samples from tumors grown for 5 weeks in vivo using IHC staining (FIG. 2b). An intense staining for uPAR expression was found, thus confirming the result from flowcytometry.

A group of mice were scanned 15 hr post injection with ICG-Glu-Glu-AE105 in the IVIS Lumina XR. A high uptake in the tumor was observed (FIG. 3) and quantitative analysis of the tumor and background uptake, resulted in a tumor-to-background (TBR) ratio of 3.52±0.167 (n=10) (FIG. 4a). The max radiance for the tumors was in the range 3.43E+08±0.34E+08 radiance efficiency.

Next, a group of mice were imaged with only ICG in order to validate the specificity of the new probe. No specific uptake was seen in the tumor. TBR for ICG was 1.04±0.04 (n=10) (The max radiance for the tumors were in the range 7.51E+06±3.13E+05). All tumors from both groups of mice were subsequently resected after the last scan and the uPAR expression in the tumor lysate was analysed. uPAR expression level was identical in each group (3.19±0.59 for ICG and 2.64±0.28 for ICG-Glu-Glu-AE105) (FIG. 4a).

Finally, to delineate the translational use of this method, the group of mice injected with ICG-Glu-Glu-AE105 was also imaged with the clinically approved camera Fluobeam® (FIG. 5). Clear tumor identification was possible due to high uptake of ICG-Glu-Glu-AE105 as seen in FIG. 5. This imaging modality gave similar TBR (3.58±0.29.) as the IVIS Lumina XR and thus confirms the translational potential of ICG-Glu-Glu-AE105.

Data Interpretation

Intraoperative optical imaging with NIR is a new emerging technique that can help surgeons remove solid tumours with higher accuracy and decrease the number of patients with positive margins. In this study, the newly synthesized probe ICG-Glu-Glu-AE105 was characterized in vitro and in vivo in a human glioblastoma xenograft mouse model.

Many designs of optical probes have been constructed. Several groups have investigated probes targeting the EGFR receptor[9], integrin αᵥβ₃ [10] and HER1 and HER2 [11]. Numerous probes are based on antibodies as targeting vectors because of the ease of conjugating them to fluorophores and the well-known high affinity for the target. However, a number of limitations in using antibodies for in vivo optical imaging are present. The size of an antibody influences the pharmacological profile, and result in a long plasma half-life which again results in a high background and decrease the potential TBR value. An acceptable TBR value is therefore only achievable 1-3 days after injection [9, 12], thus limiting the clinical usefulness and thereby the translation potential. If smaller peptides are used an optimal imaging timepoint can get as low as 3-6 hours after injection as a result of faster clearing time. In the present study, a scan time 15 hrs post injection was found to be optimal for the peptide-based probe, thus providing a clinical useful application where a patient would be injected in the evening before planned surgery the next day.

The conjugated fluorophore is also an important choice to make. There exist numerous fluorophores in the NIR window with different properties. It was chosen to use ICG since it is the most often-used fluorophore because of its long history in angiographies, It is FDA approved and has a well-established safety profile, thus paving the way for a more easy clinical translation. The fluorescent properties of ICG has been passed by other upcoming fluorophores such as IRDye 800CW. This newer developed fluorophore exhibit features as higher brightness, easier conjugation and hydrophilicity. Especially the hydrophobicity of ICG seems to be an important feature considering the reduction in binding affinity found in this study due to conjugation of ICG, where both the size and high hydrophobicity seems to be responsible for this observation. One potential solution to this observation could be to use a longer linker and/or a more hydrophilic linker such as PEG. This approach has been done with success by others [13]. However, the limited safety profile and no clinical data for IRDye 800CW in contrast to ICG, makes any clinical translation difficult in near future. Translation of a new probe from preclinical studies to the clinical bed is with an approved fluorophore as ICG more advantageous. However the linker is not only for protection of the peptide. Several studies [13] have shown that conjugation of ICG to an antibody decrease the fluorescent signal from ICG. A comparison of ICG and ICG-Glu-Glu-AE105 showed a 2-fold decrease in fluorescence intensity for the conjugated probe (data not shown). A group have though shown that quenching of ICG is eliminated when the probe interact with cells [11], due to internalization and degradation of the conjugated vector. The ICG molecule is released and de-quenched. This property can be exploited in vivo where the non-internalized circulating probe has lower fluorescence intensity than the targeted internalized probe. ICG have primarily been used for delineating malignant glioblastomas. However, ICG has only been used in excessive doses were macroscopic colouration of the tissue have delineated the tumour and the fluorescent properties have been neglected. Further, this delineation of the tumour is most likely a result of the EPR effect and not a tumour specific accumulation.

Several targets for optical imaging in cancer detection have been investigated and both endogenous and exogenous fluorophores has shown great potential for clinical translation. Conversion of 5-ALA to PpIX, an endogenous fluorescent process, has been shown to occur in excess in glioblastomas and have reached clinical studies with convincing results. An advantage uPAR, as target, holds over 5-ALA is the information given regarding the tumours phenotype. uPAR has been correlated with a poor prognosis and aggressive metastatic behavior. Further uPAR have shown to be expressed in the invasive front of the tumor and in the surrounding stroma. This makes uPAR an ideal target for NIR intraoperative optical resection of solid tumors. In addition, the receptor needs to be over expressed on the surface of the cancer cells. This has been confirmed by flowcytometry for the glioblastoma cell line used in this human xenograft model.

The main aim was to develop a targeted ICG probe, with high affinity and specificity towards uPAR and high in vivo stability. Results from this study have shown that the newly developed probe ICG-Glu-Glu-AE105 possesses all these properties. Conjugated to the clinical approved fluorophore ICG the use of this probe in intra-operative imaging has a high clinical translation potential.

### FURTHER EMBODIMENTS OF THE INVENTION

The present invention is directed to a fluorophore labelled uPAR-targeting peptide conjugate comprising an efficient combination of the type of peptide and fluorophore included in the conjugate.

Therefore, according to the present invention there is provided a fluorophore labelled uPAR-targeting peptide conjugate comprising
a fluorophore capable of detection either directly or indirectly in an optical imaging procedure;
a peptide binding to the receptor; and
a linker group which covalently links the fluorophore to the peptide binding to the receptor, said linker group either being part of the peptide binding to the receptor or being a separate component of the uPAR (urokinase Plasminogen Activator Receptor)-targeting conjugate;
   wherein the peptide comprises or is selected from:
-Asp-Cha-Phe-(D)Ser-(D)Arg-Tyr-Leu-Trp-Ser(−);
-Asp-Cha-Phe-(D)Ser-(D)Arg-Tyr-Leu-Trp-Ser-OH; or
-Asp-Cha-Phe-(D)Ser-(D)Arg-Tyr-Leu-Trp-Ser-N H₂,
   and wherein the fluorophore is selected from any of ICG, Methylene blue, Protoporphyrin IX, IRDye800CW, ZW800-1, Cy5, Cy7, Cy5.5, Cy7.5, IRDye700DX, Alexa fluor 488, Fluorescein isothiocyanate, Flav7, CH1055, Q1, Q4, H1, IR-FEP, IR-BBEP, IR-E1, IR-FGP, or IR-FTAP,
   and pharmaceutically acceptable salts thereof.

According to one embodiment of the present invention, the fluorophore labelled uPAR-targeting peptide conjugate does not comprise the compound where the fluorophore is ICG and the peptide AE105 (Asp-Cha-Phe-(D)Ser-(D)Arg-Tyr-Leu-Trp-Ser-OH).

According to one embodiment, the fluorophore is a near-infrared I fluorophore selected from the group consisting of ICG, Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate.

According to yet another embodiment, the fluorophore is a near-infrared II fluorophore selected from the group consisting of Flav7, CH1055, Q1, Q4, H1, IR-FEP, IR-BBEP, IR-E1, IR-FGP, IR-FTAP.

Moreover, according to one embodiment, the fluorophore is IRDye800CW. Furthermore, according to yet another embodiment of the present invention, the linker group is connected by covalent bonds, wherein the linker group comprises oligoethylene glycols or other short oligomers such as oligo-glycerol, oligo-lactic acid or carbohydrates which are optionally connected by covalent bonds to at least one amino acid. Moreover, the linker group may be connected by covalent bonds and wherein the covalent bonds are selected from the group consisting of an amide, a carbamate, thiourea, an ester, ether, amine, a triazole or any other covalent bond commonly used to couple chemical moieties by solid-phase synthesis.

According to one preferred alternative, the fluorophore labelled uPAR-targeting peptide conjugate has the formula

[chemical structure]

According to one embodiment, the fluorophore is a near-infrared I fluorophore or a near-infrared II fluorophore, and wherein the fluorophore has a NIR-light absorption in the range of 700-1200 nm, 700-950 nm (NIR-I), or 1000-1200 nm (NIR-II).

According to yet another embodiment, the fluorophore is a near-infrared I fluorophore or a near-infrared II fluorophore, and wherein the fluorophore has a NIR-light emission in the range of 700-1200 nm, 700-950 nm (NIR-I), or 1000-1200 nm (NIR-II).

Furthermore, with reference to some specific peptide alternatives, according to one embodiment, the fluorophore labelled uPAR-targeting peptide conjugate comprises a receptor binding peptide selected from AE105 with the sequence DChaFsrYLWS-OH, AE344 with the sequence EE-O2Oc-O2Oc-DChaFsrYLWS-OH, AE345 with the sequence EE-O2Oc-O2Oc-DChaFsrYLWS-NH₂, AE346 with the sequence O2Oc-O2Oc-DChaFsrYLWS-OH, AE347 with the sequence EE-O2Oc-DChaFsrYLWS-N H₂, AE348 with the sequence E-O2Oc-DChaFsrYLWS-NH₂, AE349 with the sequence EE-DChaFsrYLWS-OH, the sequence ICG-EE-DChaFsrYLWS-OH or AE353 with the sequence IRDye800CW-EE-O2Oc-O2Oc-DChaFsrYLWS-OH. These are further mentioned and disclosed below in e.g. table 1.

Moreover, in relation to the amino acid sequence written in another format, the following apply with reference to that part of the peptides:
AE105: Asp Cha Phe D-Ser D-Arg Tyr Leu Trp Ser-OH;
AE344: Glu Glu O2Oc O2Oc Asp Cha Phe D-Ser D-Arg Tyr Leu Trp Ser-OH,
AE345: Glu Glu O2Oc O2Oc Asp Cha Phe D-Ser D-Arg Tyr Leu Trp Ser-NH₂,
AE346: O2Oc O2Oc Asp Cha Phe D-Ser D-Arg Tyr Leu Trp Ser-OH;
AE347: Glu Glu O2Oc Asp Cha Phe D-Ser D-Arg Tyr Leu Trp Ser-NH₂;
AE348: Glu O2Oc Asp Cha Phe D-Ser D-Arg Tyr Leu Trp Ser-NH₂;
AE349: Glu Glu Asp Cha Phe D-Ser D-Arg Tyr Leu Trp Ser-OH;

### Detailed Description of the Drawings and Examples

SPR Experiments

Covalent immobilization of purified human prouPA^{S356A}—was accomplished by injecting 12.5 μg/ml protein dissolved in 10 mM sodium acetate (pH 5.0) over a CM5 chip that had been pre-activated with NHS/EDC (N-ethyl-N′-[3-diethylamino)propyl]-carbodiimide), aiming at a surface density of >5000 resonance units (RU) corresponding to 100 fmols/mm². After coupling the sensorchip was deactivated with 1 M ethanolamine. Binding of purified human uPAR as analyte was measured from 4 nM to 0.25 nM at 20° C. using 10 mM HEPES, 150 mM NaCl, 3 mM EDTA (pH 7.4) containing 0.05% (v/v) surfactant P20 as running buffer at a flow rate of 50 μl/min. In between cycles the sensorchip was regenerated by two consecutive 10-μl injections of 0.1 M acetic acid/HCl (pH 2.5) in 0.5 M NaCl. The inhibition of 3-fold dilutions of the compounds in question was measured for 4 nM uPAR with identical running conditions. All experiments were performed on a BiacoreT200 instrument.

Results

For each inhibition peptide inhibition profile of uPAR binding to immobilized uPA there has been run a preceding standard curve and all calculations are based on the that standard curve. Table 1 summarizes the results.

TABLE 1

| | | | IC₅₀ uPAR |
|---|---|---|---|
| | Sequence | IC₅₀ uPAR wt | H47C-N259C |
| AE105 | DChaFsrYLWS-OH | 7.8 ± 1.0 nM | 4.5 ± 1.5 μM |
| AE344 | EE-O2Oc-O2Oc- | 5.7 ± 0.5 nM | — |
| | DChaFsrYLWS-OH | | |
| AE345 | EE-O2Oc-O2Oc- | 31.8 ± 1.5 nM | — |
| | DChaFsrYLWS-NH₂ | | |
| AE346 | O2Oc-O2Oc-DChaFsrYLWS-OH | 16.1 ± 0.9 nM | — |
| AE347 | EE-O2Oc-DChaFsrYLWS-NH₂ | 3.5 ± 0.1 nM | — |
| AE348 | E-O2Oc-DChaFsrYLWS-NH₂ | 6.7 ± 0.2 nM | — |
| AE349 | EE-DChaFsrYLWS-OHICG-EE-DChaFsrYLWS- | 12.5 ± 0.6 nM | — |
| | OH | 142 ± 13 nM | 0.99 ± 0.05 μM |
| AE353 | IRDye800CW-EE-O2Oc-O2Oc-DChaFsrYLWS- | 20.0 ± 1.1 nM | 5.8 ± 0.02 μM |
| | OH | | |

It is clear from table 1 that a second generation of uPAR targeting peptides have been generated, that provides an improvement over the former provides alternatives. In contrast to the ICG derivative, IRDye800CW variant of AE344 (AE353) both targets the wt uPAR with high affinity and show low affinity towards a constrained uPAR variant (negative control). By expanding the hydrophilic linker region, a product with much better solubility properties has been obtained and the original high affinity of the parent peptide (AE105) has been maintained despite having tethered a large reporter group to its N-terminus (IRDye800CW).

Biochemistry and Optical Properties The present invention describes the synthesis of uPAR-targeting fluorescent probe based on a uPAR-targeting peptide conjugate, IRDye800CW-AE344, with the molecular structure shown in FIG. 6. The binding properties to uPAR was preserved yielding an IC₅₀=20 nM±1.1 nM (SD) for the competition on the binding of the natural ligand urokinase-type plasminogen activator (FIG. 7).

The vis/NIR spectral properties showed an abortion peak at λ_{abs,max}=777 nm (FIG. 8) and a slightly right shifted excitation profile with an excitation peak at λ_{excitation,max}=784 nm. The fluorescence emission spectrum showed peak emission at λ_{emission,max}=794 nm resulting in a Stokes shift of 10 nm. Photostability revealed a preserved fluorescence intensity of 84% after continuous laser exposure for 1 h and of 62% after 2 h (FIG. 9).

In Vivo Cancer Imaging Specificity

In one example of the present invention fluorescent probe IRDye800CW-AE344 was submitted to in vivo cancer imaging. In visual light the orthotopic GBM was non-visible through the intact brain but was clearly visualized on NIR imaging (FIG. 10). Additionally, on cross sectioned brain the tumor extent was visible with clear demarcation from healthy tissue allowing distinction between tumor tissue and healthy brain tissue. Histological assessment reveled co-localization of the tumor extent on H&E staining, the NIR microscopy, and on uPAR stained immunohistochemistry (FIG. 11) demonstrating that the optical probe of the present invention truly targets the biomarker/tumor with high sensitivity (all tumor is fluorescent) and high specificity (all fluorescent signal is tumor tissue).

For fluorescence-guided surgery (FGS), the surgeon relies on clear identification (signal intensity) and distinction (TBR). The higher dose of 12 nmol revealed a similarly high TBR of 6.7 but at the expense of both delayed peak time of 15 h and a decreased tumor MFI at 58% of that at 6 nmol. Thus, the ideal probe and the optimal dose should lead to both a high intensity and a high TBR.

Compared to prior art, the uPAR-targeting peptide conjugate provides with an improved water solubility, higher signal intensity, and increased TBR. Hence, the fluorescent probe of the present invention safely visualizes GBM with a high TBR of above 4.5 from 1 h to 12 h after injection of 6 nmol that allows for flexible use and complies perfectly with the standard workflow at surgical departments where the probe can be injected shortly prior to surgery as soon as an intravenous access is established e.g. at the preparation for surgery/induction of anesthesia. The useful time-window will be reached when surgery begins and persist throughout even long operations. The highest TBR of 7.0 was observed 3 h post injection of 6 nmol with a high absolute signal intensity. Further, a prolonged incubation time, with the intention to give the fluorescent probe time to clear from circulation and localize in the tumor, would be highly impractical and does not comply with the established clinical workflow and requires the patient to come for an extra visit several days prior to the operation. Also, it is not uncommon that surgery is postponed or cancelled with short notice.

Dynamic Imaging

In another example, dynamic imaging of orthotopic GBM on cross sections revealed clear tumor visualization at all four doses (1, 3, 6 and 12 nmol). The highest TBR (7.0) was observed 3 h after injection of a dose of 6 nmol (FIG. 12). The other doses tested showed maximal TBRs of 4.4 (1 nmol), 6.6 (3 nmol), and 6.7 (12 nmol) at 0.5 h, 1 h, and 15 h, respectively (FIG. 13). The corresponding tumor mean fluorescence intensities (MFI) were 29, 77, 82 and 48 (a.u.), for 1, 3, 6 and 12 nmol doses, respectively (333 ms exposure time). Hence, it was observed a correlation between fluorescence intensity and dose with increasing intensity (both tumor and background) with increasing dose (FIG. 14). MFI in both tumor and background were highest at the time of injection and decreased continuously over time. Interestingly, at 6 nmol the TBR initially increased from 4.7 at 1 h to 7.0 at 3 h followed by a decrease to 6.1 and 4.5 at 6 h and 12 h, respectively. In the same time there was a continuous decrease in tumor MFI with 108 (a.u.) at 1 h to 82 (a.u.) at 3 h corresponding to a 24% decrease (FIG. 15). The background MFI at the same time points decreased from 24 (a.u.) to 11 (a.u.) corresponding to a 54% decrease and the increase in TBR was thus a result of a higher background clearance rate compared to the tumor clearance rate between 1 h to 3 h.

In Vivo Binding Specificity

Competitively blocking and administration of control ligand (IRDye800CW-AE354, non-binding, scrambled peptide) in animals with orthotopic GBM showed a lower signal intensity compared to the active probe (FIG. 16). The normalized TBR values for the groups receiving active, blocked or scrambled probe were 1.00 (reference value), 0.70 (p=0.006), and 0.52 (p=0.001), respectively (FIG. 17).

Pharmacokinetics and Toxicology

At 1 h the kidneys exhibited the highest MFI of 1,236 (a.u.) followed by the lungs, skin, and liver of 101 (a.u.), 99 (a.u.), and 88 (a.u.), respectively. In comparison, the tumor exhibited a signal of 65 (a.u.) and the brain of 19 (a.u.) (FIG. 19). The biodistribution showed accumulation primarily in the skin and the kidneys and the normalized uptake in major organs were skin=100 (reference), kidneys=38.8, lungs=4.2, heart=0.4, spleen=0.4, liver=12.2, pancreas=3.4, colon=4.0, small intestine=11.2, ventricle 0.9, brain=0.7, tumor=0.1. The signal in the small intestine was limited to the proximal intestines/intestinal content and only modest signal was seen in the distal part (FIG. 18 and also shown in FIG. 22A).

In FIG. 22 there is shown biodistribution and acute toxicity. All data is at 1 h post injection. In 22A, as in FIG. 18, there is shown images of organs representing the fluorescence intensity and biodistribution of 3 nmol IRDye800CW-AE344 (exposure time: 333 ms). The arrow at the small intestines indicates the proximal end. Kidneys saturated and thus out of range at the color calibration bar. In 22B there is shown liver histology (H&E stained). Moreover, in 22C there is shown kidney histology (H&E stained). Histologically, the liver tissue was normal with lobular configuration without inflammation, fibrosis, cholestasis or deposits. The kidney tissue was normal with preserved glomeruli and tubules without atrophy, inflammation or fibrosis.

Quantification of fluorescence signal (the data are normalized with the skin as the reference) is shown in 22D. In 22E there is shown plasma stability quantified as area under curve (AUC) on HPLC, normalized to 0 hours. The probe was stable within the relevant time window with normalized area under curve (AUC) values of 73% and 67% intact probe after 6 and 12 hours, respectively un murine plasma, and 61% and 43% intact probe after 6 and 12 hours, respectively in human plasma.

Plasma Stability

1,600 ul human and murine plasma were separately incubated with 2.4 nmol IRDye800-AE344 in 10 ul PBS at 37° C. in dark. 200 ul samples were collected at 0, 0.5, 1, 2, 3, 6, 12 and 24 hours. Plasma proteins were precipitated by addition of 200 ul acetonitrile and the samples were centrifuged at 10,000 G for 10 min. The supernatant was collected for analysis. The supernatant from time zero from both the human and mouse serum was analyzed to establish the retention time of the intact IRDye800CW-AE344 on HPLC-MS on a RSLC Dionex Ultimate 3000 (Thermo) instrument coupled to a QTOF Impact HD. The column was an Aeris widepore 3.6 μm C4 column (150×4.6 mm, Phenomenex) and the solvent system was solvent A: water containing 0.1% Formic acid; solvent B: acetonitrile containing 0.1% formic acid. Method: 0-1 min 5% solvent B, 1-18 min 5%-50% solvent B with a flowrate of 1 mL/min. This showed that the intact molecule was eluded after 14 minutes. The method, column, and solvents were then transfer to another Dionex Ultimate 3000 (Thermo) which had a 3100-FLD fluorescent detector that employed 774 nm as excitation wavelength and measured the emission at 798 nm. All samples were then run using the settings above and the AUC at the 14 min peak was used to calculate the degradation with 0 h for both murine and human plasma as the reference.

Fluorescence-Guided Resection

Preoperatively, the tumor was visible on WL and NIR images (FIG. 20 upper panel). The surgeon performed the resection only assisted by WL until the surgeon considered all tumor tissues was removed. The surgical bed was then evaluated by FLI and the NIR signal revealed residual tumor tissue that was not identified and removed in WL (FIG. 20 middle panel). Assisted by NIR, the surgeon identified additional tumor tissue and resected it until no or very little NIR signal was visible indicating complete tumor resection (FIG. 20 lower panel). A video of fluorescence-guided surgery is also available in the online supplementary materials.

As should be understood from above, in FIG. 20 there is shown fluorescence-guided surgery (6 nmol IRDye800CW-AE344 at 3 h) performed with the EleVision™ IR system. Upper panel: Images were acquired prior to surgery. Middle panel: Pictures were acquired following surgery in white light and clearly visualizes remaining tumor tissue. The signal is more intense compared to upper panel and is due to the fact that the remaining tumor is now more exposed. Lower panel: Images were acquired at the end of the fluorescence-guided surgery and the fluorescent tissue is completely removed indicating complete tumor resection.

In Vivo Binding Specificity

Competitive blocking of IRDye800CW-AE344 binding with AE120 (the dimer version of the AE105) and administration of the inactive non-targeting version IRDye800CW-AE354 in orthotopic GBM showed lower signal intensity compared to the active probe (see FIGS. 21A and 21B). In FIG. 21A there is shown NIR images of cross sectioned brains at 1 h after injection of (left to right): 3 nmol IRDye800CW-AE344 (active), 3 nmol IRDye800CW-AE344+1.7 mg AE120 (the dimer version of the AE105) (blocked), and 3 nmol IRDye800CW-AE354 (binding inactive). Images are contrast enhanced equally with the scale bar representing the true values. In FIG. 21B mean fluorescence intensities for tumor and background are shown.

The corresponding normalized TBR values for the groups receiving active, blocked or inactive probe were 6.6, 4.6 (p=0.012), and 3.4 (p=0.0025), respectively (see FIG. 21C).

### Experimental

Biochemistry, Optical Properties and Binding Specificity

A tumor targeting NIR probe was developed by conjugating the IRDye®800CW fluorophore (LI-COR) with a small uPAR targeting peptide AE105.

The peptide was produced by Fmoc solid-phase peptide synthesis on an automated peptide synthesizer (Biotage® Syro Wave) using a Fmoc Ser(t-Bu) TentaGel S PHB 0.25 mmol/g resin. Subsequently, 5 mg of IRDye®800CW was conjugated to the peptide by HATU/HOAT coupling.

The crude probe was purified by a 3-step process on RP-HPLC (on a Dionex Ultimate 3000 system with a fraction collector). Step 1: Preparative C18 column (Phenomenex Gemini, 110 Å 5 μm C18 particles, 21×100 mm), solvent A, water+0.1% TFA, solvent B: acetonitrile+0.1% TFA. Gradient elution (0-5 min: 5%; 5% to 60% 5-32 min) at flow rate 15 mL/min. The fractions containing the fluorescent probe were freeze dried. Step 2: Preparative C4 column (Phenomenex Jupiter, 300 Å 5 μm C18 particles, 21×100 mm), solvent A: water+0.1% TFA, solvent B: Methanol+0.1% TFA. Gradient elution (0-5 min: 5%; 5% to 60% 5-32 min) at flow rate 15 mL/min. The fractions containing the fluorescent probe were freeze dried. Step 3: Preparative C18 column (Phenomenex Gemini, 110 Å 5 μm C18 particles, 21×100 mm), solvent A: water+0.1% TFA, solvent B: acetonitrile+0.1% TFA. Gradient elution (0-5 min: 5% to 30; 30% to 40% 5-40 min) at flow rate 15 mL/min. The fractions containing the fluorescent probe were freeze dried. The product was verified by mass spectrometry and the purity was evaluated by 2-step analytical RP-HPLC.

Fluorophore excitation and emission profiles were obtained with PTI QuantaMaster 400 (Horiba Ltd., Japan). Excitation profile was measured at λₑₘᵢₛₛᵢₒₙ=850 nm and the emission profile were measured at λ_{excitation}=740 nm with xenon arc lamps as excitation source. Absorption was measured on a Cary 300 UV-Vis (Agilent, Santa Clara, Calif., USA).

The photostability was evaluated by a factor 2 dilution series with four samples from 0.23-1.8 nM IRDye800-AE344 in 100 μL phosphate buffered saline (PBS) placed in a black 96-well plate. The well was placed in a black box with the Fluobeam mounted in the top at a distance of 23 cm from the well plate. Image acquisition was performed at 0 min, 10 min, 15 min, 20 min, 0.5 h, 1 h, 2 h, 3 h, 6 h, 10 h, 15 h, 24 h. Each dilution sample was normalized to time point 0 and the data from all four samples were pooled.

Surface plasmon resonance (SPR) was applied to determine the 1050-value of IRDye800-AE344 on the uPAR⋅uPA interaction in solution using a Biacore 3000 instrument essentially as described. In brief; pro-uPA^{S356A}, which is the natural ligand for uPAR. It is produced recombinantly with the active site S356A mutated so that it has no enzymatic activity (thus, in S356A the active site Ser is replaced by an inactive Ala), was immobilized on a CM5 sensor chip (immobilizing >5000 RU ˜0.1 pmol pro-uPA/mm²) providing a very high surface density of pro-uPA. This results in a heavily mass transport limited reaction causing the observed association rates (v_{obs}) to be directly proportional to the concentrations of binding active uPAR in solution—given only low concentrations of uPAR are tested (here 0.06 nM to 2 nM). The analysis was carried out by measuring vobs of a fixed uPAR concentration (2 nM) incubated with a 3-fold dilution series of IRDye800-AE344 (ranging from 0.076 nM to 1.5 μM) for 300 sec at 20° C. with a flow rate of 50 μL/min. A standard curve was measured in parallel (2-fold dilution of uPAR covering 0.06 nM to 2 nM) including one repeated concentration point at the end to validate the biological integrity of the sensor chip. Running buffer contained 10 mM HEPES, 150 mM NaCl, 3 mM EDTA and 0.05% (v/v) surfactant P20, pH 7.4. The sensor chip was regenerated with two injections of 0.1 M acetic acid, 0.5 M NaCl. The parent nonamer peptide antagonist AE105 (Asp-Cha-Phe-D-Ser-D-Arg-Tyr-Leu-Trp-Ser-OH) was analyzed in parallel as positive control and the closed uPAR^{H47C-N259C} was used as negative control as its uPA binding cavity cannot accommodate AE105.

Cell Line and Culturing.

U-87 MG-luc2 cells (Caliper, Hopkinton, Mass., USA) were cultured in Dulbecco's Modified Eagle's medium (DMEM)+GlutaMAX added 10% fetal bovine serum (FBS) and 1% Penicillin-Streptomycin at 37° C. in humid 5% CO2 air. The cells were passaged or harvested when reaching 80-90% confluency.

Animal Models

All experimental procedures in animals were carried out in accordance with the approval by The Animal Experiments Inspectorate, Denmark. 7-10-week-old female nude mice (strain: Rj:NMRI-Foxn1nu/nu, JANVIER LABS, France) were orthotopically xenografted with U-87 MG-luc2 cells.

Prior to any surgical procedure, the animals were anesthetized with Hypnorm (0.315 mg/ml fentanyl, 10 mg/ml fluanisone)+Midazolam (5 mg/ml)+sterile water in the ratio 1:1:2 by subcutaneous injection of 0.01 ml/g body weight of the solution.

The orthotopic GBM tumor model was established by inoculation of 500.000 cells in 10 μL ice cold PBS in the right hemisphere (1.5 mm lateral and 0.5 mm posterior to the bregma at 2 mm depth) using a stereotaxic frame (KOPF INSTRUMENTS, Tujunga, Calif., USA) with the automated microinjection pump UMP3 (WPI, Sarasota, Fla., USA). The cells were injected over 5 min with the syringe kept in place for further 3 min before retraction. Tumor growth was subsequently monitored on MRI with the BioSpec 7T (Bruker, Billerica, Mass., USA) with axial and coronal T2-wighted sequences. When the tumor size reached 2-17 mm³, the animals were included in the fluorescence imaging protocol.

Fluorescence Imaging

All image acquisition was performed with the Fluobeam system with (Fluosoft version: 2.2.1) (Fluoptics, Grenoble, France). To characterize the in vivo biodistribution and tumor imaging properties, IRDye800-AE344 was administered through tail vein injection into all the GBM bearing mice (n=35) at four different doses: 1 nmol, 3 nmol, 6 nmol, and 12 nmol. Two mice were sacrificed for each time point and the brain was removed and cross sectioned through the tumor for imaging. Preliminary testing revealed slower background clearance for the higher doses prolonging the time window. Accordingly, image acquisition was performed at following time points after injection:
1 nmol: 0.5 h, 1 h, and 2 h
3 nmol: 1 h, 2 h, 3 h, and 5 h
6 nmol: 1 h, 3 h, 6 h, and 12 h
12 nmol: 1 h, 3 h, 5 h, 10 h, 15 h, and 24 h

The target specificity to uPAR was evaluated by two different methods: competitive blocking with the uPAR targeting peptide AE120 ((DChaFsrYLWSG)₂-βAK) and IRDye800 conjugated to a scrambled peptide, AE354 (IRDye800CW-Glu-Glu-NH—CH₂—CH₂—O—CH₂—CH₂—O—CH₂—CO—NH—CH₂—CH₂—O—CH₂—CH₂—O—CH₂—CO-Asp-Cha-Glu-(D)Ser-(D)Arg-Tyr-Leu-Glu-Ser-OH) with similar peptide length as the active peptide. The competitive blocking dose of 1.4-2.8 mg AE120 was injected intraperitoneally 15-30 min prior to intravenous injection of 3 nmol IRDye800CW-AE344 and imaged at 1 h (n=5). The scrambled peptide was administered at 3 nmol and the animals were imaged after 1 h (n=4).

Biodistribution was assessed in animals (n=2) receiving 3 nmol IRDye800-AE344 and was euthanized after 1 h for organ dissection and imaging (exposure time: 333 ms). Due to renal excretion, the fluorescence signal in the kidneys were out of scale compared to all the other organs. Thus, images were acquired at a lower exposure time of 40 ms and the signal intensity was subsequently extrapolated to be comparable to the other organs. The skin was imaged partially (1.25 g) and the biodistribution was extrapolated with respect to the full weight of the skin (4.9 g).

Image Processing and Analysis

Images were analyzed and processed in ImageJ 1.52a, NIH, USA. Signal measurements were performed on the raw images generated by the Fluoptics system. Tumor signal was measured as a mean fluorescence intensity of the whole tumor and background signal was measured as the mean of a representative area with no tumor on the contralateral hemisphere. Presented pictures are contrast enhanced in ImageJ with the Contrast Enhancement (Saturated pixels: 0.3%).

Pathological Assessment

Pathological assessment was used to evaluate the colocalization of the fluorescence signal and cancer cells (sens+spec). The cross sectioned brain specimens from the fluorescence imaging were either paraffin embedded for H&E and IHC staining, or cryostat sectioned for fluorescence microscopy. Paraffin embedding was performed by fixation in 4% formalin for 24 h following suspension in ethanol and subsequent paraffine embedding. The embedded tissue was axially sectioned into xx um thick slices and stained. IHC staining was performed with an in-house antibody, poly-rabbit-anti-human-uPAR, produced by Finsen Laboratory, Rigshospitalet (Copenhagen, Denmark) and H&E staining was performed by common standard procedure. The stained slides were imaged with the ZEISS Axio Scan.Z1 slide scanner (Carl Zeiss, Oberkochen, Germany).

Cryostat sectioning was performed by fixation of the specimen in Tissue-Tek O.C.T. on dry ice. The fixed tissue was sliced axially and mounted on slides for immediate fluorescence imaging.

Liver and kidneys: Tissue was formalin fixed and paraffin embedded. Sections of 2-4 um thick were cut and a routine staining panel applied including: H&E, modified sirius, PAS and Masson trichome for both and additional PAS with silver for kidneys and PAS with diastase, iron, reticulin artisan and oxidised orcein for the livers. Livers were immunohistochemically stained, immunohistochemical evaluation on 3 μm thick sections was done using the CK7 antibody from Dako/Agilent, GA619 (clone OV-TL12/30) following the manufacturer's instructions. The staining took place on the Omnis from Agilent utilizing the EnVision Flex+ detection kit (GV800). The primary antibody was diluted using Antibody Diluent (Dako DM830) and were incubated for 20 minutes. The sections were counterstained with hematoxylin.

Materials and Procedure for Peptide Synthesis

Materials

IRDye800-Glu-Glu-O2Oc-O2Oc-Asp-Cha-Phe-D-ser-D-arg-Tyr-Leu-Trp-Ser-OH. All other materials were obtained from commercial suppliers; Fmoc Ser(t-Bu) TentaGel S PHB 0.25 mmol/g, was from Rapp Polymere GmbH. All Amino acids were Fmoc Na-amino protected and carried side-chain protecting groups: tert-butyl (Ser, Asp, Glu and Tyr), tert-butyloxycarbonyl (Boc, for Trp), 2,2,4,6,7-pentamethyl-dihydrobenzofuran-5-sulfonyl (Pbf, for Arg). Fmoc-O2Oc-OH Fmoc-[2-(2-aminoethoxy)ethoxy]acetic acid N,N-dimethylformamide (DMF), N-methylpyrrolidone (NMP),N-[(1H-benzotriazol-1-yl)(dimethylamino)methylene]-N-methylmethanaminium hexafluorophosphate N-oxide (HBTU), 1-Hydroxy-7-azabenzotriazole (HOAt), trifluoroacetic acid (TFA), piperidine and N,N-diisopropylethylamine (DIPEA) were from Iris Biotech GmbH, while methanol, acetonitrile, formic acid, triethylsilane (TES), dichloromethane (DCM), 1-[Bis(dimethylamino)methylene]-1H-1,2,3-triazolo[4,5-b]pyridinium 3-oxide hexafluorophosphate, N-[(Dimethylamino)-1H-1,2,3-triazolo-[4,5-b]pyridin-1-ylmethylene]-N-methylmethanaminium hexafluorophosphate N-oxide (HATU) were from Sigma-Aldrich. IRDye®800CW Carboxylate from LI-COR.

Peptide Synthesis

The peptide was produced by Fmoc solid-phase peptide synthesis on an automated peptide synthesizer; Biotage® Syro Wave. The synthesis was carried out on a Fmoc Ser(t-Bu) TentaGel S PHB 0.25 mmol/g resin, using 0.1 mmol scale.

The NºFmoc deprotection was performed at room temperature (RT) in two stages by treating the resin with piperidine/DMF (2:3) solution for 3 min, followed by piperidine/DMF (1:4) solution for 15 minutes. The resin was then washed with NMP (×3), DCM (×1), and then NMP (×2). All couplings of amino acids used 4 eq. of amino acid and O2Oc spacer, 4 eq. of HOAt, 3.9 eq. of HBTU, and 7.4 eq. of DIEA in NMP. The coupling time was 60 minutes at RT. All couplings were repeated to ensure maximum incorporation, and after the second coupling the resin was washed with NMP (×4).

When all amino acids and O2Oc spacer was attached and the last Fmoc group was removed, a test cleavage was performed, which showed that the purity was above 80%, calculated from the LC-MS chromatogram.

In order to attach the fluorophore to the synthesized peptide, 5 mg of IRDye®800CW Carboxylate was dissolved in 1 ml DMF. To this 2 mg of HATU, 1 mg HOAT and 1.7 μl DIEA was added. The solution was shaken for 5 min and then transferred to 100 mg of resin with the synthesized peptide and left to react for 12 hours in darkness. After end reaction the resin was washed with DMF(×5) and DCM(×6). Then the peptide was cleaved from the resin using 95% TFA; 5% Water with a 2 hour reaction time. The TFA was removed with nitrogen flow. The peptide was then precipitated in cold diethyl ether.

Purification

The crude peptide was purified by a 3 step process on RP-HPLC (on a Dionex Ultimate 3000 system with a fraction collector). First the peptide was purified on a preparative C18 column (Phenomenex Gemini, 110 Å 5 μm C18 particles, 21×100 mm) using the following solvent system: solvent A, water containing 0.1% TFA; solvent B, acetonitrile containing 0.1% TFA. Gradient elution (0-5 min: 5%; 5% to 60% 5-32 min) was applied at a flow rate of 15 mL min⁻¹. The fractions containing the fluorescent peptide were freeze dried. They were purified using the following conditions on step 2: preparative C4 column (Phenomenex jupiter, 300 Å 5 μm C18 particles, 21×100 mm) using the following solvent system: solvent A, water containing 0.1% TFA; solvent B, Methanol containing 0.1% TFA. Gradient elution (0-5 min: 5%; 5% to 60% 5-32 min) was applied at a flow rate of 15 mL min⁻¹. The fractions containing the fluorescent peptide were freeze dried. And then final step of the purification performed on a preparative C18 column (Phenomenex Gemini, 110 Å 5 μm C18 particles, 21×100 mm) using the following solvent system: solvent A, water containing 0.1% TFA; solvent B, acetonitrile containing 0.1% TFA. Gradient elution (0-5 min: 5% to 30; 30% to 40% 5-40 min) was applied at a flow rate of 15 mL

Analysis

Peptide purity was established using 2 different analytical methods performed on UHPLC-MS on a RSLC Dionex Ultimate 3000 (Thermo) instrument coupled to a QTOF Impact HD. During the first method an Aeris 3.6 μm widepore C4 column (50×2.1 mm, Phenomenex) with a flow rate of 0.5 mL/min was used with the following solvent system: solvent A, Water containing 0.1% Formic acid; solvent B, Methanol containing 0.1% Formic acid. The column was eluted using a linear gradient from 5%-75% of solvent B.

The second method involved kinetex 2.6 μm EVO 100 Å C18 column (50×2.1 mm, Phenomenex) with a flow rate of 0.5 mL/min. The following solvent system was used: solvent A, Water containing 0.1% Formic acid; solvent B, acetonitrile containing 0.1% Formic acid. The column was eluted using a linear gradient from 5%-100% of solvent B. The synthesis yielded 2 mg of 98% pure peptide. Chemical Formula: C₁₂₉H₁₇₃N₁₈O₄₁S₄. Calculated Mass 2758.0888; found: [M+2H]²⁺1380.0523; [M+3H]³⁺920.3723; [M+4H]⁴⁺

### YET FURTHER EMBODIMENTS OF THE INVENTION

Below there is provided yet further embodiments of the present invention. The embodiments below are linked to another aspect of the peptide conjugate concept according to the present invention, namely the pharmacokinetic profile thereof.

In line with this, according to one embodiment of the present invention, the fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again, and preferably wherein the fluorophore labelled uPAR-targeting peptide conjugate is a fluorophore labelled human uPAR-targeting conjugate.

According to yet another embodiment of the present invention, the fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again, preferably wherein the fluorophore labelled uPAR-targeting peptide conjugate is a fluorophore labelled human uPAR-targeting peptide conjugate.

Furthermore, according to yet another embodiment of the present invention, wherein the plasma half-life is maximum 75 hours, preferably maximum 20 hours, more preferably maximum 15 hours, more preferably in the range of 6-15 hours, most preferably in the range of 6-10 hours.

Moreover, according to yet another embodiment of the present invention, the fluorophore labelled uPAR-targeting peptide conjugate has a pharmacokinetic profile where a TBR (tumor-to-background ratio) of at least 2.8 is reached within 3.5 hours post administration and where a level of TBR of at least 2.8 is held during at least 30 minutes before decreasing again.

Furthermore, according to yet another embodiment, a peak TBR of the fluorophore labelled uPAR-targeting conjugate after administration is at least 3.

Moreover, according to one embodiment of the present invention, receptor binding affinity of the fluorophore labelled uPAR-targeting peptide conjugate to uPAR, defined as Kd, is maximum 2,500 nM, preferably maximum 2,000 nM, more preferably maximum 500 nM, most preferably in a range of 2,000-300 nM.

According to yet another embodiment of the present invention, the speed of which the protein (P)-ligand (L) complex takes place may be defined as

$$P + L {{⇌}^{{K}_{on}}}_{{K}_{off}} P \cdot L ,$$

where Kₒₙ is a constant of the binding reaction and where K_{off} is a constant for the dissociation of the protein-ligand complex, and wherein Kₒₙ>1×10³ and/or K_{off}<1×10⁻¹ s⁻¹, more preferably wherein Kₒₙ≥7.3×10⁵ M⁻¹ s⁻¹ (M being molar, i.e. concentration) Moreover, according to yet another embodiment, Kₒₙ of the fluorophore labelled uPAR-targeting peptide conjugate is equal to or higher than that of uPA being the natural ligand, implying Kₒₙ≥4.6×10⁶

Moreover, according to one embodiment of the present invention, the fluorophore labelled uPAR-targeting peptide conjugate displaces the natural ligand (uPA) binding to uPAR with an IC₅₀ value which is maximum 1,000 nM, preferably maximum 200 nM, more preferably maximum 50 nM, most preferably maximum 25 nM.

Furthermore, according to yet another embodiment of the present invention, the uPAR-targeting conjugate has a sensitivity for detection of cancer tissue of at least 60%, preferably above 70%, more preferably above 80% and most preferably above 90%.

In one preferred embodiment, the fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again, wherein the plasma half-life is maximum 15 hours,

wherein Kₒₙ of the fluorophore labelled uPAR-targeting peptide conjugate is equal to or higher than that of uPA being the natural ligand, implying Kₒₙ≥4.6×10⁶ M⁻¹ s⁻¹, and wherein the fluorophore labelled uPAR-targeting peptide conjugate is a fluorophore labelled human uPAR-targeting peptide conjugate.

As mentioned, the suitable dosage range is 0.1-2,000 mg per dosage unit. In line with this, according to one embodiment, the concentration of the fluorophore labelled uPAR-targeting peptide conjugate according to claim 1, is in the range of 0.1-2,000 mg per dosage unit, preferably in the range of 1-1,000 mg per human dosage unit.

Moreover, the present invention also refers to an optical imaging method comprising the steps of:

(i) administering of the fluorophore labelled uPAR-targeting peptide conjugate according to the present invention, to a target tissue,
(ii) allowing time for the fluorophore labelled uPAR-targeting peptide conjugate to accumulate in the target tissue and establishing a receptor binding, after administration into the human or animal body;
(iii) illuminating the target tissue with light of a wavelength absorbable by the fluorophore; and
(iv) detecting fluorescence emitted by the fluorophore and forming an optical image of the target tissue.

Furthermore, a peptide conjugate according to the present invention, may also photothermic capabilities. In lie with this, according to yet another embodiment, the present invention is directed to a fluorophore labelled uPAR-targeting peptide conjugate according to the present invention, wherein the fluorophore labelled uPAR-targeting peptide conjugate comprises a photothermic agent capable of absorbing light that is transformed into heat upon irradiation with an external source of light and capable of being detected either directly or indirectly in an optical imaging procedure. Moreover, as a further part of this aspect, the present invention also refers to a method of optical imaging of cancer of a subject involving administering a fluorophore labelled uPAR-targeting peptide conjugate according to above to the subject and generating an optical image of at least a part of the subject to which said compound has distributed, wherein the fluorophore labelled uPAR-targeting peptide conjugate comprises a photothermic agent capable of absorbing light that is transformed into heat upon irradiation with an external source of light and capable of being detected either directly or indirectly in an optical imaging procedure, said method also comprising irradiation with an external light source that activates the photothermic agent, preferably with an external laser source, more preferably near infrared light.

The photothermic agent e.g. may be a fluorescent agent selected from NIR-1 group fluorophores, e.g. ICG, Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate; or may be a fluorescent agent selected from the NIR-II group fluorophores, such as Flav7, CH1055, Q1, Q4, H1, IR-FEP, IR-BBEP, IR-E1, IR-FGP, IR-FTAP.

### REFERENCES

1. Pogue B W, Gibbs-Strauss S L, Valdes P A, et al. Review of Neurosurgical Fluorescence Imaging Methodologies. IEEE J Select Topics Quantum Electron 16:493-505. doi: 10.1109/JSTQE.2009.2034541
2. Mushawah M D Catherine M Appleton M D Amy E Cyr M D William E Gillanders M D Rebecca L Aft M D PhD Timothy J Eberlein M D Feng Gao PhD Julie A Margenthaler M D Al JABF, Mushawah M D Al F, MD CMA, et al. (2012) Positive margin rates following breast-conserving surgery for stage I-Ill breast cancer: palpable versus nonpalpable tumors. Journal of Surgical Research 177:109-115. doi: 10.1016/j.jss.2012.03.045
3. Nguyen Q T, Tsien R Y (2013) Fluorescence-guided surgery with live molecular navigation—a new cutting edge. Nature Publishing Group 1-10. doi: 10.1038/nrc3566
4. Vahrmeijer A L, Hutteman M, van der Vorst J R, et al. (2013) Image-guided cancer surgery using near-infrared fluorescence. Nature Publishing Group 10:507-518. doi: 10.1038/nrclinonc.2013.123
5. Persson M, Madsen J, Østergaard S, et al. (2012) Quantitative PET of human urokinase-type plasminogen activator receptor with 64Cu-DOTA-AE105: implications for visualizing cancer invasion. Journal of Nuclear Medicine 53:138-145. doi: 10.2967/jnumed.110.083386
6. Persson M, Madsen J, Østergaard S, et al. (2012) 68Ga-labeling and in vivo evaluation of a uPAR binding DOTA- and NODAGA-conjugated peptide for PET imaging of invasive cancers. Nuclear Medicine and Biology 39:560-569. doi: 10.1016/j.nucmedbio.2011.10.011
7. Persson M, Liu H, Madsen J, et al. (2013) First 18F-labeled ligand for PET imaging of uPAR: In vivo studies in human prostate cancer xenografts. Nuclear Medicine and Biology 40:618-624. doi: 10.1016/j.nucmedbio.2013.03.001
8. Li Z B, Niu G, Wang H, et al. (2008) Imaging of Urokinase-Type Plasminogen Activator Receptor Expression Using a 64Cu-Labeled Linear Peptide Antagonist by microPET. Clinical Cancer Research 14:4758-4766. doi: 10.1158/1078-0432.CCR-07-4434
9. Day K E, Sweeny L, Kulbersh B, et al. (2013) Preclinical Comparison of Near-Infrared-Labeled Cetuximab and Panitumumab for Optical Imaging of Head and Neck Squamous Cell Carcinoma. Mol Imaging Biol. doi: 10.1007/s11307-013-0652-9
10. Hutteman M, Mieog J S D, van der Vorst J R, et al. (2011) Intraoperative near-infrared fluorescence imaging of colorectal metastases targeting integrin &alpha;v&beta;3 expression in a syngeneic rat model. YEJSO 37:252-257. doi: 10.1016/j.ejso.2010.12.014
11. Ogawa M, Kosaka N, Choyke P L, Kobayashi H (2009) In vivo Molecular Imaging of Cancer with a Quenching Near-Infrared Fluorescent Probe Using Conjugates of Monoclonal Antibodies and Indocyanine Green. Cancer Res 69:1268-1272. doi: 10.1158/0008-5472. CAN-08-3116
12. Ogawa M, Regino C A S, Seidel J, et al. (2009) Dual-Modality Molecular Imaging Using Antibodies Labeled with Activatable Fluorescence and a Radionuclide for Specific and Quantitative Targeted Cancer Detection. Bioconjugate Chem 20:2177-2184. doi: 10.1021/bc900362k
13. Sano K, Nakajima T, Miyazaki K, et al. (2013) Short PEG-Linkers Improve the Performance of Targeted, Activatable Monoclonal Antibody-Indocyanine Green Optical Imaging Probes. Bioconjugate Chem 24:811-816. doi: 10.1021/bc400050k
14. Li Y, Rey-Dios R, Roberts D W, et al. (2014) Peer-Review Reports. World Neurosurgery 1-11. doi: 10.1016/j.wneu.2013.06.014
15. Henricus J. M. Handgraaf, Floris P. R. Verbeek, et al. (2014) Real-time near-infrared fluorescence guided surgery in gynecologic oncology: A review of the current state of the art. Gynecologic Oncology http://dx.doi.org/10.1016/j.ygyno.2014.08.005

1. A fluorophore labelled uPAR-targeting peptide conjugate comprising
a fluorophore capable of detection either directly or indirectly in an optical imaging procedure;
a peptide binding to the receptor; and
a linker group which covalently links the fluorophore to the peptide binding to the receptor, said linker group either being part of the peptide binding to the receptor or being a separate component of the uPAR (urokinase Plasminogen Activator Receptor)-targeting conjugate;
wherein the peptide comprises or is selected from:
-Asp-Cha-Phe-(D)Ser-(D)Arg-Tyr-Leu-Trp-Ser(−);
-Asp-Cha-Phe-(D)Ser-(D)Arg-Tyr-Leu-Trp-Ser-OH; or
-Asp-Cha-Phe-(D)Ser-(D)Arg-Tyr-Leu-Trp-Ser-NH₂,
and wherein the fluorophore is selected from any of ICG, Methylene blue, Protoporphyrin IX, IRDye800CW, ZW800-1, Cy5, Cy7, Cy5.5, Cy7.5, IRDye700DX, Alexa fluor 488, Fluorescein isothiocyanate, Flav7, CH1055, Q1, Q4, H1, IR-FEP, IR-BBEP, IR-E1, IR-FGP, or IR-FTAP,
and pharmaceutically acceptable salts thereof.

2. The fluorophore labelled uPAR-targeting peptide conjugate according to claim 1, wherein the fluorophore labelled uPAR-targeting peptide conjugate does not comprise the compound where the fluorophore is ICG and the peptide AE105 (Asp-Cha-Phe-(D)Ser-(D)Arg-Tyr-Leu-Trp-Ser-OH).

3. The fluorophore labelled uPAR-targeting peptide conjugate according to claim 1, wherein the fluorophore is a near-infrared I fluorophore selected from the group consisting of ICG, Methylene blue, Protoporphyrin IX, IRDye800CW, ZW800-1, Cy5, Cy7, Cy5.5, Cy7.5, IRDye700DX, Alexa fluor 488, Fluorescein isothiocyanate.

4. The fluorophore labelled uPAR-targeting peptide conjugate according to claim 1, wherein the fluorophore is a near-infrared II fluorophore selected from the group consisting of Flav7, CH1055, Q1, Q4, H1, IR-FEP, IR-BBEP, IR-E1, IR-FGP, IR-FTAP.

5. The fluorophore labelled uPAR-targeting peptide conjugate according to claim 1, wherein the fluorophore is IRDye800CW.

6. The fluorophore labelled uPAR-targeting peptide conjugate according to claim 1, wherein the linker group is connected by covalent bonds, wherein the linker group comprises oligoethylene glycols or other short oligomers such as oligo-glycerol, oligo-lactic acid or carbohydrates which are optionally connected by covalent bonds to at least one amino acid.

7. The fluorophore labelled uPAR-targeting peptide conjugate according to claim 1, wherein the linker group is connected by covalent bonds and wherein the covalent bonds are selected from the group consisting of an amide, a carbamate, thiourea, an ester, ether, amine, a triazole or any other covalent bond commonly used to couple chemical moieties by solid-phase synthesis.

8. The fluorophore labelled uPAR-targeting peptide conjugate according to claim 1, having the formula

[chemical structure]

9. The fluorophore labelled uPAR-targeting peptide conjugate according to claim 1, wherein the fluorophore is a near-infrared I fluorophore or a near-infrared II fluorophore, and wherein the fluorophore has a NIR-light absorption in the range of 700-1200 nm, 700-950 nm (NIR-I), or 1000-1200 nm (NIR-II).

10. The fluorophore labelled uPAR-targeting peptide conjugate according to claim 1, wherein the fluorophore is a near-infrared I fluorophore or a near-infrared II fluorophore, and wherein the fluorophore has a NIR-light emission in the range of 700-1200 nm, 700-950 nm (NIR-I), or 1000-1200 nm (NIR-II).

11. The fluorophore labelled uPAR-targeting peptide conjugate according to claim 1, wherein the fluorophore labelled uPAR-targeting peptide conjugate comprises a receptor binding peptide selected from AE105 with the sequence DChaFsrYLWS-OH, AE344 with the sequence EE-O2Oc-O2Oc-DChaFsrYLWS-OH, AE345 with the sequence EE-O2Oc-O2Oc-DChaFsrYLWS-NH₂, AE346 with the sequence O2Oc-O2Oc-DChaFsrYLWS-OH, AE347 with the sequence EE-O2Oc-DChaFsrYLWS-NHz, AE348 with the sequence E-O2Oc-DChaFsrYLWS-NH₂, AE349 with the sequence EE-DChaFsrYLWS-OH, the sequence ICG-EE-DChaFsrYLWS-OH or AE353 with the sequence IRDye800CW-EE-O2Oc-O2Oc-DChaFsrYLWS-OH.

12. The fluorophore labelled uPAR-targeting peptide conjugate according to claim 1, wherein fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again, and preferable wherein the fluorophore labelled uPAR-targeting peptide conjugate is a fluorophore labelled human uPAR-targeting conjugate.

13. The fluorophore labelled uPAR-targeting peptide conjugate according to claim 1, wherein the fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again, preferably wherein the fluorophore labelled uPAR-targeting peptide conjugate is a fluorophore labelled human uPAR-targeting peptide conjugate.

14. The fluorophore labelled uPAR-targeting peptide conjugate according to claim 1, wherein the plasma half-life is maximum 75 hours, preferably maximum 20 hours, more preferably maximum 15 hours, more preferably in the range of 6-15 hours, most preferably in the range of 6-10 hours.

15. The fluorophore labelled uPAR-targeting peptide conjugate according to claim 1, wherein the fluorophore labelled uPAR-targeting peptide conjugate has a pharmacokinetic profile where a TBR (tumor-to-background ratio) of at least 2.8 is reached within 3.5 hours post administration and where a level of TBR of at least 2.8 is held during at least 30 minutes before decreasing again.

16. The fluorophore labelled uPAR-targeting peptide conjugate according to claim 1, wherein a peak TBR of the fluorophore labelled uPAR-targeting conjugate after administration is at least 3.

17. The fluorophore labelled uPAR-targeting peptide conjugate according to claim 1, wherein receptor binding affinity of the fluorophore labelled uPAR-targeting peptide conjugate to uPAR, defined as Kd, is maximum 2,500 nM, preferably maximum 2,000 nM, more preferably maximum 500 nM, most preferably in a range of 2,000-300 nM.

18. The fluorophore labelled uPAR-targeting peptide conjugate according to claim 1, wherein the speed of which the protein (P)-ligand (L) complex takes place may be defined as
$$P + L {{⇌}^{{K}_{on}}}_{{K}_{off}} P \cdot L ,$$
where Kₒₙ is a constant of the binding reaction and where K_{off} is a constant for the dissociation of the protein-ligand complex, and wherein Kₒₙ>1×10³ M⁻¹ s⁻¹ and/or K_{off}<1×10⁻¹ s⁻¹, more preferably wherein Kₒₙ 7.3×10⁵ M⁻¹ s⁻¹.

19. The fluorophore labelled uPAR-targeting peptide conjugate according to claim 1, wherein Kₒₙ of the fluorophore labelled uPAR-targeting peptide conjugate is equal to or higher than that of uPA being the natural ligand, implying Kₒₙ≥4.6×10⁶ M⁻¹ s⁻¹.

20. The fluorophore labelled uPAR-targeting peptide conjugate according to claim 1, wherein the fluorophore labelled uPAR-targeting peptide conjugate displaces the natural ligand (uPA) binding to uPAR with an IC₅₀ value which is maximum 1,000 nM, preferably maximum 200 nM, more preferably maximum 50 nM, most preferably maximum 25 nM.

21. The fluorophore labelled uPAR-targeting peptide conjugate according to claim 1, wherein the uPAR-targeting conjugate has a sensitivity for detection of cancer tissue of at least 60%, preferably above 70%, more preferably above 80% and most preferably above 90%.

22. The fluorophore labelled uPAR-targeting peptide conjugate according to claim 1, wherein the fluorophore labelled uPAR-targeting peptide conjugate has a pharmacokinetic profile where a TBR (tumor-to-background ratio) of at least 2.5 is reached within 3.5 hours post administration and where a level of TBR of at least 2.5 is held during at least 30 minutes before decreasing again,
wherein the plasma half-life is maximum 15 hours, wherein Kₒₙ of the fluorophore labelled uPAR-targeting peptide conjugate is equal to or higher than that of uPA being the natural ligand, implying Kₒₙ≥4.6×10⁶ M⁻¹ s⁻¹, and wherein the fluorophore labelled uPAR-targeting peptide conjugate is a fluorophore labelled human uPAR-targeting peptide conjugate.

23. A pharmaceutical composition comprising the fluorophore labelled uPAR-targeting peptide conjugate according to claim 1, wherein the concentration of the fluorophore labelled uPAR-targeting peptide conjugate according to claim 1, is in the range of 0.1-2,000 mg per dosage unit, preferably in the range of 1-1,000 mg per human dosage unit.

24. An optical imaging method comprising the steps of:
(i) administering of the fluorophore labelled uPAR-targeting peptide conjugate according to claim 1, to a target tissue,
(ii) allowing time for the fluorophore labelled uPAR-targeting peptide conjugate to accumulate in the target tissue and establishing a receptor binding, after administration into the human or animal body;
(iii) illuminating the target tissue with light of a wavelength absorbable by the fluorophore; and
(iv) detecting fluorescence emitted by the fluorophore and forming an optical image of the target tissue.

25. A fluorophore labelled uPAR-targeting peptide conjugate according to claim 1, wherein
the fluorophore labelled uPAR-targeting peptide conjugate comprises a photothermic agent capable of absorbing light that is transformed into heat upon irradiation with an external source of light and capable of being detected either directly or indirectly in an optical imaging procedure.

26. A method of optical imaging of cancer of a subject involving administering a fluorophore labelled uPAR-targeting peptide conjugate according to claim 26 to the subject and generating an optical image of at least a part of the subject to which said compound has distributed, wherein the fluorophore labelled uPAR-targeting peptide conjugate comprises a photothermic agent capable of absorbing light that is transformed into heat upon irradiation with an external source of light and capable of being detected either directly or indirectly in an optical imaging procedure,
said method also comprising irradiation with an external light source that activates the photothermic agent, preferably with an external laser source, more preferably near infrared light.
